(19) United States
(12) Patent Application Publication
(10) Pub. No.: US 2026/0215695 A1
(43) Pub. Date: Jul. 30, 2026

(54) ADAPTIVE SERVO GAIN CONTROL FOR MULTIWAVELENGTH VOLUME CLAMP

(71) Applicant: Becton, Dickinson and Company, Franklin Lakes, NJ (US)

(72) Inventor: ILJA GUELEN, Franklin Lakes, NJ (US)

(21) Appl. No.: 19/147,614

(22) PCT Filed: Jan. 12, 2024
(86) PCT No.: PCT/US2024/011383

§ 371 Date: Jul. 11, 2025

Related U.S. Application Data
(60) Provisional application No. 63/479,721, filed on Jan. 12, 2023.

(51) Int. Cl. A61B 5/0225; A61B 5/021; A61B 5/026; A61B 5/0295; A61B 5/1455; G16H 40/63

(52) U.S. Cl. CPC A61B 5/02255; A61B 5/02141; A61B 5/0261; A61B 5/0295; A61B 5/14552; G16H 40/63; A61B 2560/0223

(57) ABSTRACT

A blood characteristic sensing system includes a light emitter, a light sensor, and a pressurizable cuff. The system encircles a sensing region of a patient appendage with the pressurizable cuff, emitting multiple discrete wavelengths of light into the sensing region and sensing amplitudes for each wavelength received through the patient appendage. A sensed plethysmogram value reflecting arterial volume is generated based on these sensed amplitudes, and pressurization of the cuff is modulated to partially clamp arterial volume within the sensing region via a closed-loop control algorithm based on the sensed plethysmogram value and a setpoint plethysmogram value. Differential absorption of the sensed wavelengths is used to produce a composition of analysis of arterial blood within the sensing region, and arterial blood pressure is sensed based on pressurization of the cuff required to partially clamp arterial volume.

### CROSS-REFERENCE TO RELATED APPLICATIONS

The current application claims priority under 35 U.S.C. 119 (e) to U.S. Provisional Application No. 63/479,721, entitled “ADAPTIVE SERVO GAIN CONTROL FOR MULTIWAVELENGTH VOLUME CLAMP” to Guelen et al., filed Jan. 12, 2023, the disclosure of which is incorporated herein by reference in its entirety.

### BACKGROUND

The present disclosure relates generally to blood characteristic sensing, and more particularly to gain control for volume clamping in a multi-function, multi-wavelength arterial blood pressure and blood composition sensor.

Some non-invasive arterial blood pressure sensors generate a pressure reading by clamping (i.e. holding constant) arterial volume within a sensing region such as a portion of a finger surrounded by a pressurizable cuff. Such systems directly assess arterial volume, e.g. optically, increasing or decreasing constriction applied by the pressurizable cuff via closed-loop control to compensate for fluctuations in arterial volume caused by the pulsation of blood. The resulting clamping pressure serves as a proxy for or estimate of the arterial blood pressure waveform (AP), allowing blood pressure to be non-invasively monitored over long time periods, without interruption. In some examples, the arterial volume waveform can also be directly analyzed to estimate mean arterial blood pressure, diastolic pressure, or systolic pressure.

Some non-invasive blood composition sensors estimate characteristics such as blood oxygen saturation and hemoglobin composition based on differential absorption of a spectrum of wavelengths of light by the blood during arterial pulsation.

### SUMMARY

This disclosure presents a method of operating a blood characteristic sensing system. The sensing system includes a light emitter, a light sensor, and a pressurizable cuff. The system is operated by encircling a sensing region of a patient appendage with the pressurizable cuff, emitting multiple discrete wavelengths of light from the light emitter into the sensing region of the patient appendage, and sensing light amplitudes received by the light sensor, from the light emitter, through the patient appendage, for each of the discrete wavelengths of light. A sensed plethysmogram value reflecting arterial volume within the sensing region is generated based on the sensed light amplitudes, and pressurization of the pressurizable cuff is modulated to partially clamp arterial volume within the sensing region via a closed-loop control algorithm responsive to a closed-loop error value reflecting a difference between the sensed plethysmogram value and a setpoint plethysmogram value. A composition analysis of arterial blood within the sensing region is then generated based on differential absorption of the multiple discrete wavelengths of light, as sensed by the light sensor during arterial pulsation within the sensed region. A sensed arterial blood pressure is generated based on the pressurization of the pressurizable cuff required to partially clamp the arterial volume.

This disclosure also presents a non-invasive sensor system including a pressurizable cuff, a light emitter, a light sensor, a compositional analysis module, and a controller. The pressurizable cuff is pressurized via a metered fluid supply and sized to encircle a patient appendage and define a sensing region. The light emitter is anchored to the pressurizable cuff and configured to emit light through the sensing region of the patient appendage at multiple discrete wavelengths. The light sensor is also anchored to the pressurizable cuff, is positioned to receive light emitted by the light emitter, and is configured to generate a sensed plethysmogram signal based on this received light. The compositional analysis module is configured to assess blood composition within the sensing region based on differential absorption of the discrete wavelengths emitted by the light emitter, as detected by the light sensor, during artery pulsation within the sensing region. The controller is configured to compute an error value as a difference between the sensed plethysmogram signal and a setpoint plethysmogram value, and to operate a closed-loop control mode wherein the metered fluid supply is driven to partially clamp arteries within the sensing region based on the error value and a gain level, thereby adjusting a clamping pressure equivalent to a sensed arterial blood pressure.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a simplified perspective view of a non-invasive sensor system fitted to a human hand.

FIG. 2 is a schematic view of the non-invasive sensor system of FIG. 1 in operation.

FIG. 3 is a parallel graph of cuff pressure and overall light level as a function of time in an illustrative example of an operation period of the non-invasive sensor system of FIGS. 1 and 2.

FIG. 4 is a flowchart illustrating a method of operation of the non-invasive sensor system of FIGS. 1 and 2.

FIG. 5 is functional block diagram illustrating a control process for the non-invasive sensor system of FIGS. 1 and 2.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

The present disclosure describes an approach to volume clamping control in a multi-function sensor system. This system uses received light amplitude as a sensed plethysmogram value (hereinafter “pleth signal”) representing arterial volume within a sensed region, physically clamps arterial volume via closed-loop control based on this plethysmogram reading, and reports resulting clamping pressure as arterial blood pressure waveform. With further analysis, systolic (SYS) pressure, diastolic (DIA), and mean arterial pressure (MAP) can be derived from this waveform. Light used to generate pleth signals is emitted across multiple wavelengths, permitting analysis of blood composition (e.g. blood oxygen saturation, total hemoglobin, percentage methemoglobin, percentage carboxyhemoglobin) by comparing light absorption at multiple wavelengths during arterial pulsation. This approach allows a single non-invasive multi-function sensor to produce readings for both arterial blood pressure and arterial blood composition.

The volume clamping noted above for arterial blood pressure sensing is superficially at odds with the requirement that arterial pulsation be permitted (i.e. not clamped) to evaluate blood composition based on differential wavelength absorption. The present disclosure provides methods and systems for closed-loop clamping using adaptive gain control tailored to provide sufficient clamping to sense arterial blood pressure accurately, while nevertheless permitting sufficient arterial pulsation to enable blood composition evaluation. This balancing of clamping gain between “too high” (preventing composition analysis) and “too low” (impairing arterial blood pressure sensing accuracy) is described in detail below. The present approach modulates gain to closed-loop volume clamping control to avoid fully clamping the artery, while still clamping sufficiently to produce an arterial blood pressure reading with negligible loss of accuracy.

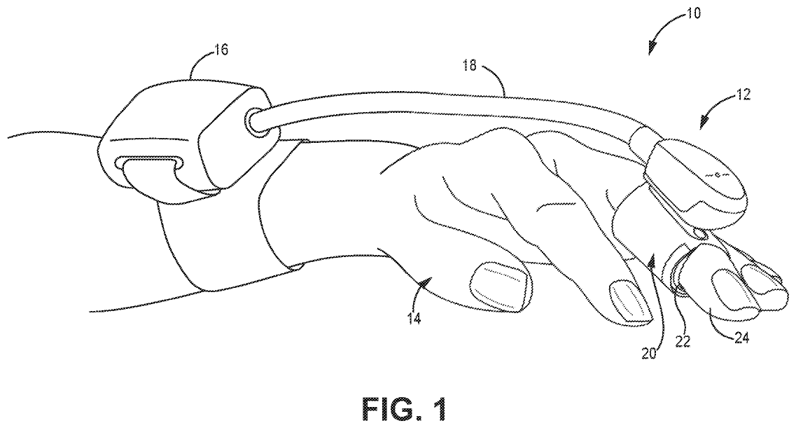

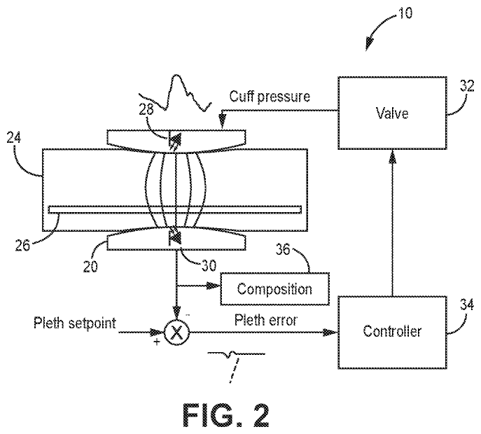

FIG. 1 provides a simplified perspective view sensor system 12 attached to hand 14. FIG. 2 is a schematic view of sensor system 12 in operation. FIGS. 1 and 2 are primarily described together. As shown in FIG. 1, sensor system 12 is a non-invasive hemodynamic sensor capable of generating arterial blood pressure measurements through volume clamping. Sensor system 12 can include housing 16, connector 18, cuff 20, and pressurizable bladder 22. In the illustrated embodiment, cuff 20 is a ring or similar structure surrounding or bracketing finger 24 of hand 14, while housing 16 is a wrist-mounted device coupled to cuff 20 via connector 18. In the most general case, however, sensor system 12 can differ substantially from the layout illustrated in FIG. 1. Sensor system 12 can, for example, include multiple separate connectors 18 between elements attached to finger 24 (e.g. cuff 20), and/or can relocate housing 16 to other locations (e.g. integrated with cuff 20, or separately disposed at a peripheral location). In the illustrated example, cuff 20 surrounds a sensing region of a finger 24 of hand 14. At least one artery 26 passes through the sensing region. Cuff 20 also anchors pressurizable bladder 22, which can for example be an expandable annular air bladder fed by an air line included within connector 18, or from another source. In the most general case, however, pressurizable bladder 22 can be any sort of mechanism suited to apply pressure to finger 24 based on control as described below. Sensor system 12 and hand 14 together make up combined physical system 10 (sometimes referred to as a plant or plant system) responsive both to changes in the patient and change in control of sensor system 12.

As shown in FIG. 2, cuff 20 includes light emitter 28 and light sensor 30. Light emitter 28 emits multiple discrete wavelengths of light through the sensing region (denoted in FIG. 2 by path lines through finger 24) for reception by light sensor 30. In the examples described in detail below, the wavelengths of light emitted by light emitter 28 can all fall within the visible-to-infrared spectrum. Light sensor 30 detects both overall received light amplitude and specific received light amplitudes at each of the discrete wavelengths emitted by light emitter 28. In some examples, light emitter 28 and light sensor 30 can be situated on opposite sides of cuff 20, such that light travels directly through the sensing region of finger 24 from light emitter 28 to light sensor 30. More generally, however, scattering of light from light emitter 28 inside tissue of finger 24 allows light emitter 28 and light sensor 30 to be effective even when not disposed on opposite sides of cuff 20, e.g. when located proximate one another.

Overall light amplitude received at light sensor 30 from transmission by emitter 28 is referred to hereinafter as pleth (plethysmogram) signal, and is used as a proxy for inverse arterial volume within the sensing region, with a reduction in received light corresponding to an increase in arterial volume. The two arteries of the finger and connected capillaries pulsate during normal blood flow, expanding (with systolic pressure) and relaxing (with diastolic pressure) in volume over the course of each heartbeat cycle. Greater arterial volume increases absorption of emitted light, reducing the fraction of emitted light received at light sensor 30.

As principally described herein, sensor system 12 clamps arterial volume within the sensing region by pneumatically pressurizable bladder 22 through actuation of valve 32, thereby modulating air pressure provided top air bladder 22 through connector 18. Although referred to herein generally as a valve, valve 32 can most generally be any sort of flow airflow metering element, such as a servo valve or piezo pump. Sensor system 12 can clamp arterial volume by any approach that applies a known pressure to the sensing region of finger 24.

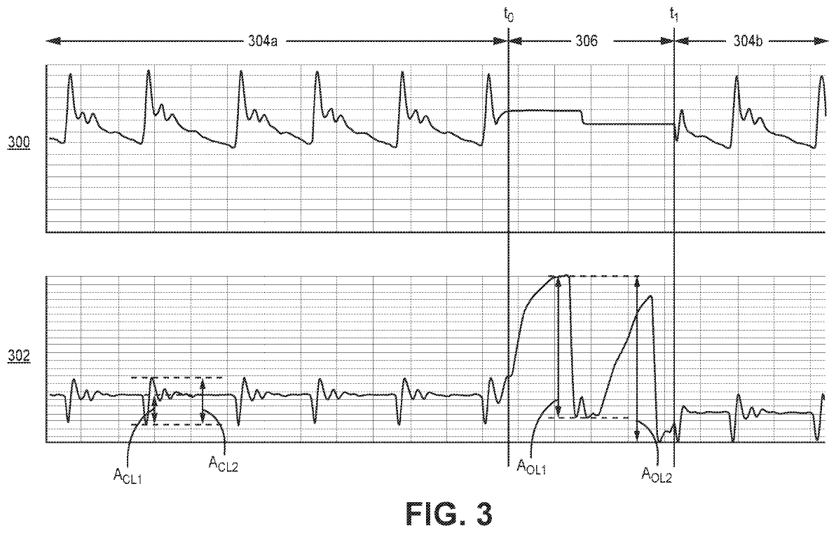

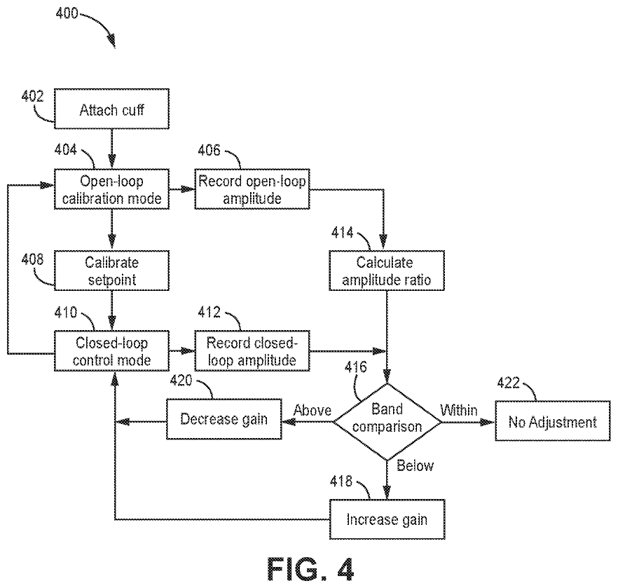

Sensor system 12 includes controller 34, with logic-capable hardware configured to adjust settings of valve 32 in a control loop responsive to the pleth signal. Controller 34 can, for example, be a control module instantiated in dedicated hardware, or a software module running on hardware within sensor system 12 or external to sensor system 12, e.g. on a communicatively connected device. Increased flow through valve 32 into pressurizable bladder 22 results in expansion of pressurizable bladder 22, physically constricting finger 24 and thereby arterial volume in the sensing region. A difference (identified hereinafter as “pleth error”) between the pleth value and a corresponding target setpoint is used as an input for control of valve 32 by controller 34. In the detailed description provided hereinafter this control scheme and method (see FIGS. 3 and 4) are described as involving proportional-integral-derivative (PID) control based on the pleth error. In the most general case, however, other forms of closed-loop control can be substituted for PID control. Controller 34 drives pressurizable bladder 22 through actuation of valve 32 to mechanically oppose changes in arterial volume within the sensing region, reducing the magnitude of arterial volume fluctuation, and keeping the arterial volume relatively constant. The valve pressure generated by this clamping process serves as a measure of arterial blood pressure. In some examples valve 32 and controller 34 can both be situated within housing 16. More generally, however, valve 32 can be located in any suitable location to meter pressurization of pressurizable bladder 22, and controller 34 can be located within housing 16, within or closer to cuff 22, or at any other location capable of supporting processing to control actuation of valve 32. In some examples, some functions of controller 34 can offloaded to a peripheral device (not shown in FIG. 1).

Light emitter 28 emits a discrete or fixed known spectrum of light across a range of wavelengths (e.g. a range of or including primarily visible-to-infrared wavelengths) within which absorption differs detectably across material compositions of interest. In one illustrative example, light emitter 28 can emit a range of wavelengths exclusively within the infrared band (700 nm to 1000 nm). In alternative examples, a broader range of light including visible and/or microwave band light can be used (e.g. 500 nm to 1100 nm). In the most general case, light emitter 28 produces known amplitudes of light at a range of wavelengths broad enough to distinguish absorption spectra associated with at least two parameters including but not limited to blood oxygen saturation, total hemoglobin, percentage methemoglobin, or percentage carboxyhemoglobin.

Composition analysis module 36 evaluates blood composition based on differential absorption of the various discrete wavelengths of light emitted by light emitter 28 during arterial pulsation, as detected by light sensor 30. Different materials such as oxyhemoglobine (O2Hb; saturated blood), deoxyhemoglobine (HHb; desaturated blood), and water, by way of example, all have detectably different absorption spectra.

In one implementation, composition analysis module 36 determines arterial oxygen saturation, which can be computed using pulse oximetry. To perform pulse oximetry, two or more discrete wavelengths of light can be utilized. In some implementations, light emitter 28 is utilized to perform pulse oximetry in addition to, or alternatively, in some implementations, light emitter 28 comprises a single emission source (e.g., diode) that can provide two or more discrete wavelengths of light (or bands of wavelength of light). In some implementations, light emitter 28 comprises at least two emission sources (e.g., at least two diodes) such that two or more discrete wavelengths of light (or bands of wavelength of light) can be emitted simultaneously. Generally, when at least two emission sources are utilized, the light emission sources can be provided near one another (e.g., within 0.5 mm) to yield similar light pathways, but any configuration can be utilized. As previously described, light emitter 28 can emit a range of wavelengths exclusively within the infrared band (700 nm to 1000 nm) or visible and/or microwave band light can be used (e.g. 500 nm to 1100 nm) to perform pulse oximetry.

Pulse oximetry relies on the fact that a portion of absorbed light signal is constant regardless of the moment in the cardiac cycle (e.g., light absorbed by the tissue, the venous blood, and the nonpulsatile arterial blood) and a portion of absorbed light signal will vary as dependent on arterial blood pulsation as related to cardiac cycles. This constant light signal is referred to as the DC signal and the variable light signal is referred to as the AC signal. Using the DC and AC signals of two wavelengths of light (λ1 and λ2), a ratio (R) can be computed as follows:

$$R = \frac{( \frac{{AC}_{λ1}}{{DC}_{λ1}} )}{( \frac{{AC}_{λ2}}{{DC}_{λ2}} )} \qquad [ Equation 1 ]$$

Ratio R can be plotted against oxygen saturation values (e.g., SpO2) as determined experimentally to yield a calibration curve. The calibration curve can be used in analysis of arterial oxygen saturation as determined utilizing a PPG fitted upon a body appendage.

Because arterial pulsation is used to distinguish between pulsatile arterial blood and other biological materials (e.g., distinguishing between AC and DC signals), composition analysis is consequently only possible during arterial pulsation. The approach set forth hereinafter modulates proportional gain of PID control at controller 34 so as to sufficiently clamp arterial volume to produce accurate and reliable measurements of arterial blood pressure based on pressure of pressurizable bladder 22 to keep arterial volume relatively constant, while permitting sufficient arterial pulsation for composition analysis module 36 to distinguish between arterial blood and other materials. In an illustrative example, the gain produced and adjusted in this way controls actuation of pressurizable bladder 22 by varying air flow through valve 32, and thereby adjusting the resulting mechanical clamping force of pressurizable bladder 22 on finger 24.

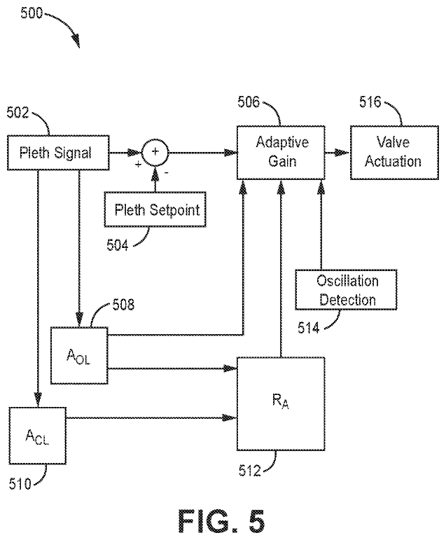

FIG. 3 is a parallel graph of cuff pressure 300 and pleth signals 302 as a function of time in an illustrative example of an operation period of sensing system 10. FIG. 3 illustrates operation of sensing system 10 through the end of first closed-loop period 304a, transitioning at time to into open-loop period 306, then into updated closed-loop period 304b at time t₁. As described above with reference to FIGS. 1 and 2, and further hereinafter with reference to FIGS. 4 and 5, sensor system 12 operates in a closed-loop mode to effectuate volume clamping of the sensing region, thereby sensing arterial blood pressure based on resulting pressurization of pressurizable bladder 22. Periods 304a and 304b are time windows within which cuff pressure is governed by this closed-loop control such that the arterial volume is kept relatively constant. In open-loop period 306, however, controller 34 evaluates the pleth value while holding the pressure of pressurizable bladder 22 constant, allowing for arterial volume dilation as it pulsates. Open-loop period 306 is used to recalibrate baseline arterial volume in the form of a pleth setpoint used in closed-loop control throughout subsequent closed-loop period 304b. This open-loop calibration period is used both for initial setup, i.e. to ascertain an initial setpoint for a new patient or cuff setup, and periodically during the monitoring of a patient to adjust for changes in patient condition. This setpoint ideally corresponds to a resting, unstressed arterial volume (i.e. undilated by arterial pulsations) within the sensing region for present conditions of finger 24, including patient hand position/posture and blood perfusion. Closed-loop volume clamping can be interrupted for open-loop calibration either on a scheduled basis to adjust for small changes in patient position or condition, and on a triggered (non-scheduled) basis in response to irregularities indicating that the current pleth setpoint requires recalibration.

FIG. 3 also provides labels to several pleth signals amplitudes referred to hereinafter with reference to FIGS. 4 and 5. Specifically, FIG. 3 illustrates first and second closed loop amplitudes A_{CL1} and A_{CL2}, respectively, and first and second open loop amplitudes A_{OL1} and A_{OL2}, respectively. First open and closed loop amplitudes A_{OL1} and A_{CL1} represent maximum fluctuation amplitudes with respect to baseline pleth signal levels. Second open and closed loop amplitudes A_{OL2} and A_{CL2} represent maximum peak-to-trough pleth signal amplitudes. As noted below, first or second amplitude values are used to constrain closed-loop control gain to permit both volume clamp-based arterial blood pressure measurement and blood composition sensing.

FIG. 4 is a flowchart illustrating ratio-based gain control method 400. Ratio-based gain control method 400 is a generalized example of a partial method of operation for sensor system 10.

Ratio-based gain control method 400 begins with attaching pressurizable bladder 22 about finger 24. (Step 402). Once pressurizable bladder 22 is in position and hand 14 is generally stationary, controller 34 engages an open-loop calibration mode (see open-loop period 306 in FIG. 3), recording a pleth value while holding cuff pressure constant. (Step 404). While in the open-loop calibration mode, controller 34 stores or records an open-loop amplitude A_{OL}, e.g. either first open-loop amplitude A_{OL1} or second open-loop amplitude A_{OL2} as described above, or both. (Step 406). The controller then adjusts or defines the pleth setpoint based on the pleth value in the open-loop calibration mode (Step 408), and enters the closed-loop control mode (see FIG. 2 and closed-loop periods 304a and 304b in FIG. 3) (Step 410), operating in this closed-loop to maintain volume clamping until the next open-loop calibration period. While in the closed-loop control mode, the controller stores or records a closed-loop amplitude A_{CL}, e.g. either first closed-loop amplitude A_{CL1} or second closed-loop amplitude A_{CL2} as described above, or both. (Step 412). In some implementations, both sets of open and closed loop amplitude can be recorded and used as alternative or comparative inputs. As mentioned above, controller 34 can cycle between open-loop and closed-loop control periodically to update and calibrate pleth setpoint, or to respond to indications that recalibration is needed. Such indications can, for example, include increased oscillation instability or increased pleth error through a set time window.

Controller 34 calculates an amplitude ratio R_{A} based on A_{CL} and A_{OL}. (Step 414). This ratio is used to determine whether gain for the closed-loop control mode (410) should be adjusted to permit both blood composition and arterial blood pressure to be sensed accurately. Specifically, controller 34 evaluates whether the calculated ratio of R_{A} falls within a permissible band. (Step 416) This band is discussed in greater detail with respect to FIG. 5. Gain at a high enough level to cause overshoots in PID control is unsuitable for both arterial blood pressure and blood composition sensing, but gain at an appropriate level for arterial blood pressure sensing can result in clamping of artery that is too aggressive to permit sufficient pulsation for accurate blood composition sensing. Conversely, if gain is at too low a level, clamping will not be sufficient to ensure reliable and accurate arterial blood pressure sensing, but would allow for adequate blood composition sensing. Accordingly, gain (e.g. overall gain, or any combination of P-, I-, or D-gain in the example of PID control) between these two extremes should be maintained at level such that detecting both arterial blood pressure and blood composition with a single multi-function sensor can be successful. To ensure success, controller 34 responds to a ratio R_{A} below the accepted band by increasing gain (Step 418) for the closed-loop control mode (Step 410), and to a ratio R_{A} above the accepted band by decreasing gain (Step 420) for the closed-loop control mode (Step 410). Gain within the accepted band requires no adjustment. (Step 422).

FIG. 5 is a functional block diagram illustrating control process 500 for sensor system 10, expanding in more concrete form upon ratio-based gain control method 400. Substantially as described with respect to closed-loop control above, controller 34 receives a pleth signal (Step 502) and a pleth setpoint (Step 504). A difference between these values is adjusted based on a variety of factors to generate an adaptive gain modification (Step 506) in the form of a multiplicative adjustment AG described in greater detail below. Specifically, controller 34 records values of both open-loop amplitude A_{OL} (step 508) and closed-loop amplitude A_{CL} (Step 510) over time, which are used to generate amplitude ratio R_{A} (Step 512).

Controller 34 adjusts the adaptive gain broadly as set forth with reference to FIG. 4 using open-loop amplitude A_{OL} and amplitude ratio R_{A}. More concretely, adaptive gain can be described as:

$$AG = GainMod / PropPletGainDivid \qquad [ Equation 2 ]$$

where AG is adaptive gain; PropPletGainDivid=C*(A_{CL}+M), where M and C can be constants; and GainMod is an adjustment factor initially set to 1.0. In some examples, values of M can be selected to permit Rₘᵢₙ to be made dependent on the state of vasoconstriction of hand 24, and can be dependent on both patent details (e.g. age and/or skin temperature) and on amplification of the hardware of the sensor and emitter, which can vary over time and/or based on circumstances. As set forth more generally above with respect to method 400, controller 34 adjusts adaptive gain AG in response to amplitude ratio R_{A} falling outside of an accepted band spanning from minimum ratio Rₘᵢₙ to maximum ratio Rₘₐₓ, where:

$${R}_{A} = ( {A}_{OL} + M ) / {AvgA}_{CL} \qquad [ Equation 3 ]$$

and AvgA_{CL} is an average value of closed loop amplitude over at least two heartbeats. To reduce the signal effect of respiration, for example, AvgA_{CL} can be an average closed loop amplitude value over eight or more patient heartbeats. Gain is adaptively driven toward a value with Rₘᵢₙ<R_{A}<Rₘₐₓ by incrementally adjusting GainMod depending upon the value of amplitude ratio R_{A}. The target band for amplitude ratio R_{A} can, for example, range from Rₘᵢₙ=5 to Rₘₐₓ=20. In a more constrained case, the band may range from Rₘᵢₙ=12 to Rₘₐₓ=14. If amplitude ratio R_{A} falls below Rₘᵢₙ in a particular evaluation cycle, GainMod is increased, e.g. such that:

$$GainMod *= A * ( {R}_{min} / {R}_{A} ) \qquad [ Equation 4 ]$$

where A is an adjustment factor greater than one, e.g. 1.05. Similarly, if amplitude ratio R_{A} rises above Rₘₐₓ in a particular evaluation cycle, GainMod is reduced, e.g. such that:

$$GainMod *= B * ( {R}_{max} / {R}_{A} ) . \qquad [ Equation 5 ]$$

where B is an adjustment factor less than one, e.g. 0.95. Adjustment factors A and B are selected at minimum to avoid overshooting desired values, i.e. preventing any correction from reducing amplitude ratio R_{A} to a new R_{A}<Rₘᵢₙ, or increasing amplitude R_{A} to a new R_{A}>Rₘₐₓ. In other illustrative examples A and B can be set to other values, e.g. 1.1 and 0.9, respectively.

Adaptive gain AG can also, in some examples, be adjusted based on detection of excessive oscillation in the pleth signal. (Step 514). In particular, controller 34 is capable of incrementally reducing Rₘᵢₙ if a level of oscillation is unacceptable-indicated, for example, by a count of PID overshoot oscillations that exceeds a threshold value (e.g. 4), to a minimum floor value of reduced Rₘᵢₙ. This floor value of reduced Rₘᵢₙ can, for example, be up to 30% less than an initial value of Rₘᵢₙ. In some examples the range of Rₘᵢₙ to Rₘₐₓ can be reduced based on a state of vasoconstriction within the sensing region. Adaptive gain AG can be limited by a fade factor that limits the magnitude by which gain can change from one process iteration to the next.

Controller 34 sets adaptive gain AG based on the aforementioned factors. To avoid rapid fluctuations in gain, controller 34 can be limited to adjust GainMod only after at least a threshold number of heartbeats (e.g. 8) have passed since a previous adjustment. This timing requirement can be waived immediately after an open-loop calibration period. The adaptive gain generated by controller 34 in step 506 drives valve actuation. (Step 516). In some examples, further control parameter processing can be included for other purposes between steps 506 and 516.

The method and apparatus set forth herein allow a single multi-function sensor to sense both arterial blood pressure and blood composition using a multi-wavelength light emitter and sensor. This approach is enabled by gain control targeting a gain band producing gain high enough to partially clamp arterial volume and thereby generate accurate and reliable arterial blood pressure readings, but low enough to retain sufficient arterial pulsation to distinguish blood composition from non-blood-related differential light absorption.

### Examples

The following are non-exclusive descriptions of possible examples for implementing the various concepts of the present disclosure.

A method of operating a noninvasive blood characteristic sensing system including a light emitter, a light sensor, and a pressurizable cuff, the method comprising: encircling a sensing region of a patient appendage with the pressurizable cuff; emitting multiple discrete wavelengths of light from the light emitter into the sensing region of the patient appendage; sensing light amplitudes received by the light sensor, from the light emitter, through the patient appendage, for each of the discrete wavelengths of light; generating a sensed plethysmogram value reflecting arterial volume within the sensing region based on the sensed light amplitudes; modulating pressurization of the pressurizable cuff in a closed-loop control mode to partially clamp arterial volume within the sensing region via a closed-loop control algorithm responsive to a closed-loop error value reflecting a difference between the sensed plethysmogram value and a setpoint plethysmogram value; generating a composition analysis of arterial blood within the sensing region based on differential absorption of the multiple discrete wavelengths of light, as sensed by the light sensor during arterial pulsation within the sensed region; and generating a sensed arterial blood pressure based on the pressurization of the pressurizable cuff required to partially clamp the arterial volume, wherein partially clamping the arterial volume within the sensing region comprises damping but not eliminating arterial pulsation, such that the generation of the composition analysis can be performed simultaneously with partially clamping the arterial volume.

The method of the preceding paragraph can optionally include, additionally and/or alternatively, any one or more of the following features, configurations and/or additional components:

A method as set forth above, further comprising: maintaining a constant pressurization of the pressurizable cuff during a recurring open-loop calibration mode; sensing an open-loop error value reflecting a difference between the sensed plethysmogram value during the open-loop calibration mode and the setpoint plethysmogram value; and calibrating the setpoint plethysmogram value based on the open-loop error value.

A method as set forth above, further comprising: calculating a ratio of maximum open-loop error value amplitude during the recurring open-loop calibration mode to maximum closed-loop error amplitude during the closed-loop control mode; and adjusting a gain of the closed-loop control algorithm to drive the calculated ratio towards a value within a preset range.

A method as set forth above, wherein the preset range is 5 to 20.

A method as set forth above, further comprising narrowing the preset range based on a state of vasoconstriction within the sensing region.

A method as set forth above, wherein the adjusting the gain of the closed-loop control algorithm comprises increasing the gain proportionally to a degree by which the calculated ratio falls below a floor of the preset range, and reducing the gain proportionally to a degree by which the calculated ratio exceeds a ceiling of the preset range.

A method as set forth above, wherein adjustment of an incremental change to the gain is capped according to a fade value.

A method as set forth above, wherein the open-loop error amplitude and the closed-loop error amplitude are each evaluated over time windows including multiple heartbeats of the patient.

A method as set forth above, wherein the time windows are selected to include at least two heartbeats of the patient.

A method as set forth above, further comprising detecting plethysmogram signal oscillations caused by overcorrection, and reducing the gain of the closed-loop control algorithm in response to the detected signal oscillations.

A method as set forth above, wherein the generation of the composition analysis occurs during both the recurring open-loop calibration mode and the closed-loop control mode.

A method as set forth above, wherein the setpoint plethysmogram value corresponds to a resting, unstressed arterial volume.

A method as set forth above, wherein the composition analysis includes an identification of at least one blood composition characteristic selected from the group consisting of blood oxygen saturation, total hemoglobin, percentage methemoglobin, percentage carboxyhemoglobin.

A non-invasive sensor system comprising: a pressurizable cuff pressurized via a metered fluid supply and sized to encircle a patient appendage and define a sensing region; a light emitter anchored to the pressurizable cuff and configured to emit light through the sensing region of the patient appendage at multiple discrete wavelengths; a light sensor anchored to the pressurizable cuff, positioned to receive light emitted by the light emitter, and configured to generate a sensed plethysmogram signal therefrom; a compositional analysis module configured to assess blood composition within the sensing region based on differential absorption of the multiple discrete wavelengths, as detected by the light sensor, during artery pulsation within the sensing region; and a controller configured to: compute an error value as a difference between the sensed plethysmogram signal and a setpoint plethysmogram value; operate a closed-loop control mode wherein the metered fluid supply is driven to partially clamp arteries within the sensing region based on the error value and gain level; and output a sensed arterial blood pressure based on a clamping pressure resulting from the closed-loop control, wherein partially clamping the arterial volume within the sensing region comprises damping but not eliminating arterial pulsation, such that the generation of the composition analysis can be performed simultaneously with partially clamping the arterial volume.

The non-invasive sensor system of the preceding paragraph can optionally include, additionally and/or alternatively, any one or more of the following features, configurations and/or additional components:

A non-invasive sensor system as set forth above, wherein the controller is additionally configured to hold a pressure of the pressurizable cuff constant during a recurring open-loop configuration mode, and to recalibrate the setpoint plethysmogram value based on a sensed difference between the setpoint plethysmogram value and the sensed plethysmogram signal as sensed during the open-loop configuration mode.

A non-invasive sensor system as set forth above, wherein the controller is additionally configured to: record a maximum amplitude of the error value during the closed-loop control mode as a closed-loop amplitude; record a maximum amplitude of the error value during the open-loop configuration mode as an open-loop amplitude; calculate a ratio of the open-loop amplitude to the closed-loop amplitude; increase the gain level of the closed-loop control mode in response to the ratio falling below a floor value; and decrease the gain level of the closed-loop control mode in response to the ratio rising above a ceiling value.

A non-invasive sensor system as set forth above, wherein the ceiling value corresponds to a maximum gain level permitting a degree of artery pulsation within the sensing region sufficient to enable assessment of blood composition.

A non-invasive sensor system as set forth above, wherein the ceiling value is approximately 20.

A non-invasive sensor system as set forth above, wherein the floor value is approximately 5.

A non-invasive sensor system as set forth above, wherein the controller is configured to set the ceiling value based on a state of patient vasoconstriction.

A non-invasive sensor system as set forth above, wherein the floor value corresponds to a minimum gain level adequate to suppress artery pulsation within the sensing region sufficiently to minimize error in the sensed arterial blood pressure.

A non-invasive sensor system as set forth above, further comprising a metering element configured to provide the metered fluid supply to the pressurizable cuff, the metering element being controlled by the controller in the closed-loop control mode and the open-loop calibration mode.

A non-invasive sensor system as set forth above, wherein the metering element is a servo valve or a piezo pump.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of operating a noninvasive blood characteristic sensing system including a light emitter, a light sensor, and a pressurizable cuff, the method comprising:
encircling a sensing region of a patient appendage with the pressurizable cuff;
emitting multiple discrete wavelengths of light from the light emitter into the sensing region of the patient appendage;
sensing light amplitudes received by the light sensor, from the light emitter, through the patient appendage, for each of the discrete wavelengths of light;
generating a sensed plethysmogram value reflecting arterial volume within the sensing region based on the sensed light amplitudes;
modulating pressurization of the pressurizable cuff in a closed-loop control mode to partially clamp arterial volume within the sensing region via a closed-loop control algorithm responsive to a closed-loop error value reflecting a difference between the sensed plethysmogram value and a setpoint plethysmogram value;
generating a composition analysis of arterial blood within the sensing region based on differential absorption of the multiple discrete wavelengths of light, as sensed by the light sensor during arterial pulsation within the sensed region; and
generating a sensed arterial blood pressure based on the pressurization of the pressurizable cuff required to partially clamp the arterial volume,
wherein partially clamping the arterial volume within the sensing region comprises damping but not eliminating arterial pulsation, such that the generation of the composition analysis can be performed simultaneously with partially clamping the arterial volume.

2. The method of claim 1, further comprising:
maintaining a constant pressurization of the pressurizable cuff during a recurring open-loop calibration mode;
sensing an open-loop error value reflecting a difference between the sensed plethysmogram value during the open-loop calibration mode and the setpoint plethysmogram value; and
calibrating the setpoint plethysmogram value based on the open-loop error value.

3. The method of claim 2, further comprising:
calculating a ratio of maximum open-loop error value amplitude during the recurring open-loop calibration mode to maximum closed-loop error amplitude during the closed-loop control mode; and
adjusting a gain of the closed-loop control algorithm to drive the calculated ratio towards a value within a preset range.

4. The method of claim 3, wherein the preset range is 5 to 20.

5. The method of claim 3, further comprising narrowing the preset range based on a state of vasoconstriction within the sensing region.

6. The method of claim 3, wherein the adjusting the gain of the closed-loop control algorithm comprises increasing the gain proportionally to a degree by which the calculated ratio falls below a floor of the preset range, and reducing the gain proportionally to a degree by which the calculated ratio exceeds a ceiling of the preset range.

7. The method of claim 6, wherein adjustment of an incremental change to the gain is capped according to a fade value.

8. The method of claim 6, wherein the open-loop error amplitude and the closed-loop error amplitude are each evaluated over time windows including multiple heartbeats of the patient.

9. The method of claim 8, wherein the time windows are selected to include at least two heartbeats of the patient.

10. The method of claim 6, further comprising detecting plethysmogram signal oscillations caused by overcorrection, and reducing the gain of the closed-loop control algorithm in response to the detected signal oscillations.

11. The method of claim 2, wherein the generation of the composition analysis occurs during both the recurring open-loop calibration mode and the closed-loop control mode.

12. The method of claim 2, wherein the setpoint plethysmogram value corresponds to a resting, unstressed arterial volume.

13. The method of claim 1, wherein the composition analysis includes an identification of at least one blood composition characteristic selected from the group consisting of blood oxygen saturation, total hemoglobin, percentage methemoglobin, percentage carboxyhemoglobin.

14. The method of claim 13, wherein the composition analysis comprises an identification of blood oxygen saturation via pulse oximetry.

15. The method of claim 14, wherein the step of generating a composition analysis of arterial blood comprises comparing absorption of the multiple discrete wavelengths of light that is constant during arterial pulsation with absorption of the multiple discrete wavelengths of light that varies due to arterial pulsation.

16. A non-invasive sensor system comprising:
a pressurizable cuff pressurized via a metered fluid supply and sized to encircle a patient appendage and define a sensing region;
a light emitter anchored to the pressurizable cuff and configured to emit light through the sensing region of the patient appendage at multiple discrete wavelengths;
a light sensor anchored to the pressurizable cuff, positioned to receive light emitted by the light emitter, and configured to generate a sensed plethysmogram signal therefrom;
a compositional analysis module configured to assess blood composition within the sensing region based on differential absorption of the multiple discrete wavelengths, as detected by the light sensor, during artery pulsation within the sensing region; and
a controller configured to:
compute an error value as a difference between the sensed plethysmogram signal and a setpoint plethysmogram value;
operate a closed-loop control mode wherein the metered fluid supply is driven to partially clamp arteries within the sensing region based on the error value and gain level; and
output a sensed arterial blood pressure based on a clamping pressure resulting from the closed-loop control,
wherein partially clamping the arteries within the sensing region comprises damping but not eliminating arterial pulsation, such that the generation of the composition analysis can be performed simultaneously with partially clamping the arterial volume.

17. The non-invasive sensor system of claim 16, wherein the controller is additionally configured to hold a pressure of the pressurizable cuff at a constant value during a recurring open-loop configuration mode, and to recalibrate the setpoint plethysmogram value based on a sensed difference between the setpoint plethysmogram value and the sensed plethysmogram signal as sensed during the open-loop configuration mode.

18. The non-invasive sensor system of claim 17, wherein the controller is additionally configured to:
record a maximum amplitude of the error value during the closed-loop control mode as a closed-loop amplitude;
record a maximum amplitude of the error value during the open-loop configuration mode as an open-loop amplitude;
calculate a ratio of the open-loop amplitude to the closed-loop amplitude;
increase the gain level of the closed-loop control mode in response to the ratio falling below a floor value; and
decrease the gain level of the closed-loop control mode in response to the ratio rising above a ceiling value.

19. The non-invasive sensor system of claim 18, wherein the ceiling value corresponds to a maximum gain level permitting a degree of artery pulsation within the sensing region sufficient to enable assessment of blood composition via the compositional analysis module.

20. The non-invasive sensor system of claim 19, wherein the ceiling value is approximately 20.

21. The non-invasive sensor system of claim 20, wherein the floor value is approximately 5.

22. The non-invasive sensor system of claim 19, wherein the controller is configured to set the ceiling value based on a state of patient vasoconstriction.

23. The non-invasive sensor system of claim 19, wherein the floor value corresponds to a minimum gain level adequate to suppress artery pulsation within the sensing region sufficiently to minimize error in the sensed arterial blood pressure.

24. The non-invasive sensor system of claim 19, wherein the assessment of blood composition includes an identification of at least one blood composition characteristic selected from the group consisting of blood oxygen saturation, total hemoglobin, percentage methemoglobin, percentage carboxyhemoglobin.

25. The non-invasive sensor system of claim 24, wherein the assessment of blood composition comprises an identification of blood oxygen saturation via pulse oximetry.

26. The non-invasive sensor system of claim 17, further comprising a metering element configured to provide the metered fluid supply to the pressurizable cuff, the metering element being controlled by the controller in the closed-loop control mode and the open-loop configuration mode.

27. The non-invasive sensor system of claim 26, wherein the metering element is a servo valve or a piezo pump.
